(19) United States
(12) Patent Application Publication
(10) Pub. No.: US 2022/0047741 A1
(43) Pub. Date: Feb. 17, 2022

(54) APPARATUS AND SYSTEM FOR ULTRAVIOLET LIGHT SANITIZATION OF MICROSCOPE EYEPIECES AND METHOD THEREFOR

(71) Applicant: J.H. Technologies, Inc., Fremont, CA (US)

(72) Inventors: John L. Hubacz, Auburn, CA (US); Samuel Miller, Folsom, CA (US)

(73) Assignee: J.H. Technologies, Inc., Fremont, CA (US)

(21) Appl. No.: 17/402,251

(22) Filed: Aug. 13, 2021

Related U.S. Application Data
(60) Provisional application No. 63/073,227, filed on Sep. 1, 2020.
(60) Provisional application No. 63/065,969, filed on Aug. 14, 2020.

(51) Int. Cl. A61L 2/10

(52) U.S. Cl. CPC A61L 2/10; A61L 2202/20; A61L 2202/11; A61L 2202/123; A61L 2202/14

(57) ABSTRACT

The device and method of the present disclosure provides for the sanitizing or disinfection of an eyepiece of an optical instrument such as a microscope without removing the microscope from its laboratory setting. The disclosed device comprises a generally cylindrical housing having an open distal end and a closed proximal end forming an interior cavity having an inner surface with a UV reflective coating and internally positioned UV-C LEDs. The open distal end is placed over an eyepiece and secured to a retention ring attached to the eyepiece, the housing and retention ring enveloping the eyepiece for irradiation by UV light. A user then may initiate an irradiation of the eyepiece via a switch which floods the interior cavity via the UV-C LEDs for disinfection of the eyepiece.

### CROSS-REFERENCE TO RELATED APPLICATIONS

This application claims the benefit of U.S. Provisional Application No. 63/065,969, filed Aug. 14, 2020, and U.S. Provisional Application No. 63/073,227, filed Sep. 1, 2020, the contents of which are expressly incorporated herein by reference.

### STATEMENT RE: FEDERALLY SPONSORED RESEARCH/DEVELOPMENT

Not Applicable

### BACKGROUND

### 1. Technical Field

The present disclosure relates generally to an apparatus and method for sanitizing microscope eyepieces. More specifically, the present disclosure includes a housing having an opening wherein the housing includes ultraviolet (UV) light emitting diodes (LED) formed internally within the housing. The housing is placed over a microscope eyepiece via the opening and secured in place over the eyepiece to irradiate the surface of the eyepiece with UV light for sanitizing purposes.

### 2. Background

Ultraviolet irradiation is an established means for disinfection and thus can be used to prevent the spread of certain infectious diseases. In or about 1877 Downes and Blunt discovered the ability of sunlight to prevent microbial growth. This and other discoveries have led to the use of man-made light sources emitting UV light for controlling microbes. By about 1930 Gates published the first analytical bacterial action spectrum demonstrating that peak effectiveness for inactivating microbes is at 265 nm, very near the 254 nm output of low-pressure mercury (Hg) germicidal lamps. These lamps may emit shortwave ultraviolet-C (UV-C, 100-280 nm) radiation known to kill or inactivate microbes by damaging their DNA. It is understood that the principal mode of inactivation occurs when the absorption of a photon forms pyrimidine dimers between adjacent thymine bases and renders the microbe incapable of replicating.

With these understandings, UV radiation from man-made light sources is commonly used to disinfect, air, water, and surfaces. For example, U.S. Pat. No. 4,786,812 issued Nov. 22, 1988 to Humphreys entitled Portable Germicidal Ultraviolet Lamp, the substance of which is incorporated herein by reference, discloses the use of UV light to kill germs. Several factors may be considered in the effectiveness of a light source to inactivate microorganisms which includes, but may not be limited to, the dose of light (intensity of the light over a period of time), the wavelength of the light source, the distance the light is away from the microorganism, and the sensitivity of the specific type of microorganism.

UV radiation water and air purification and sterilization systems are known and have a successful history of development and use. In many UV irradiation systems, the light source of UV radiation has wavelength(s) close to the absorption peaks of biologically significant molecules of DNA and proteins. The UV irradiation system may be effective to sterilize items, mediums and air to a reduced microbe condition or to a safe microbe condition by providing the irradiation magnitude of the UV light source with an effective exposure time that may be sufficient to destroy the internal biomolecular structure of bacteria, viruses, protozoa, and germs.

Hg lamps have been used as a typical tool for UV generation for disinfection and sterilization. Hg lamps, while effective, are generally large, fragile, require significant power, and the mercury element included in the light source is an element known to have toxic effects on humans and wildlife. Because of the mercury with an Hg lamp, accidental exposure to mercury in the manufacturing and use is a concern. Also, the operating life of an Hg lamp may be typically less than 10,000 hours and disposal of the units after the useful life may be problematic due to the toxic nature of mercury. Also, Hg lamps emit a broad base spectrum and may have little ability vary and control the radiation within a desired range of the spectrum. The characteristics making up Hg lamps may suit them best for stationary mounting applications and may be impractical for portable applications.

As an alternative to a classic Hg lamp, manufacturers have developed UV LEDs to generate UV light for irradiation. UV LEDs may emit UV radiation in the desired ranges for effective microbial control of infectious diseases. UV-C LEDs provide emission of UV light in the range of wavelengths between 100 nm to 280 nm. UV-C LEDs offer effective microbial control but additionally include advantages for the development of portable, low power solutions for germicidal use for emitting UV light. UV-C LEDs are small, lightweight, and may last up to 50,000 hours. In addition, UV-C LEDs offer narrow spectrums, and are power efficient making them an excellent choice for portable disinfection applications. The narrow UV-C spectrums coincide with the absorption peaks of DNA and proteins, offering more efficient sterilization then the broadband spectrum of Hg Lamps. Additionally, LEDs are small and durable and can be easily integrated into small portable devices of virtually any shape.

The background above includes information that may be useful in understanding the present disclosure. It is not an admission that any of the information provided herein is prior art or relevant to the presently claimed inventive subject matter, or that any publication specifically or implicitly referenced is prior art.

### BRIEF SUMMARY

The device and method of the present disclosure provides for the sanitizing, sterilizing or disinfection of surfaces of an eyepiece of an optical instrument such as a microscope, telescope and binoculars. For purposes of this disclosure, the use of words optical device may include devices such as a microscope, telescope or binoculars as well as any other optical devices that includes a protruding eyepiece. The disclosed device and method provides for the in-situ or on-site disinfection of an optical device eyepiece in a laboratory or field setting. Disinfection results in the killing, disabling, destroying, inactivating or rending inert an infectious agent or pathogen. Infectious agents and pathogens may include organisms and microorganisms that cause or spread disease such as viruses, bacteria, fungus and parasites.

The disclosed device is effective in disinfecting optical device eyepieces that are typically a round cylinder made of plastic or metal and incorporate sensitive optics. Surfaces and points on the eyepiece that contact a user's face, eyes and hands are a common transfer area for pathogens such as bacteria, viruses or other microorganisms that can cause disease. In addition, the eyepieces typically include ridges, indents and other non-uniform surfaces that create areas on the eyepiece that may be difficult to effectively disinfect via chemical wipes which are regularly used for this purpose. Also, eyepieces that include rubber eyecups are also difficult to clean using chemical wipes. Also, different parts and surfaces of the eyepiece may vary in type of material such as optical glass, metal, plastic and rubber surfaces which may require more than one type of cleaning method, chemical solution, or chemical wipe. The disclosed device is designed and adapted to envelope the cylindrical optical device eyepiece in such a way that an interior ultraviolet radiation source exposes all of the surfaces of the eyepiece to deliver an effective dose of UV light to render an infectious agent or pathogen inactive or harmless. The highly reflective inner surface of the disclosed device, along with the geometrics of the reflective inner walls of the device ensures that UV-C radiation is delivered at an effective target dose to all areas of the rounded surface of eyepiece. The interior reflective surface may have a smooth mirror-like finish or may also have contoured or raised geometrics patterns to effect the reflectivity.

The disclosed device is designed to be placed over the optical device eyepiece completely covering the optical, metal and plastic structures of the eyepiece while it is in place or connected to the optical device without the need to remove the eyepiece. The device is held in place on the optical device eyepiece by an adjustable retention gasket that is mounted or secured on the eye tube of the eyepiece. The gasket is adjustable to fit different diameter eyepiece eye tubes. The fixed mounting of the gasket on the eye tube ensures the device is consistently attached to the eyepiece during disinfection so that the inner reflective walls of the device are fixed at a correct distance to provide consistent, repeatable dosages to the affected surfaces of the eyepiece, and also seals the opening of the device to avoid leakage of UV light outside the device.

The disclosed device comprises a generally cylindrical housing having an open distal end and a closed proximal end forming an interior cavity having an inner surface. Within the cavity on the closed proximal end, one or more UV-C LEDs are positioned to provide UV light to the interior cavity. The UV-C LEDs are driven by electronics placed within an electronics housing that is coupled to the proximal end of the cylindrical housing. The inner surface of the cavity includes a UV reflective surface to reflect emitted UV-LED light throughout the cavity, and the curved cylindrical walls allow for distribution of UV light throughout the cavity. The open distal end is used to receive an eyepiece of an optical device such as a microscope into the interior cavity.

A user may couple the housing to a retention ring or gasket positioned on the exterior of the eyepiece to hold the housing in place resulting in the inner surface of the cavity being a fixed distance away from the side of the eyepiece to facilitate the irradiation of light onto the entire surface of the portion of the eyepiece contained in the cavity and to also seal the interior cavity from any significant light leakage outside of the eyepiece. The user may then initiate an irradiation sequence by a switch positioned on the housing via a programmable digital timer, which when activated floods the interior cavity with UV light from the one or more UV-C LEDs for a fixed period of time, for which a countdown may be displayed on the LCD number display fixed on the face of the electronics housing. Colored LED indicators positioned on the exterior of the electronics housing may display visual cues via LEDs visible from the exterior of the device, such as indications that the irradiation sequence is underway. For example, an LED may turn off during the cycle and upon completion, displaying a continuous on display, and may include a different LED color.

The disclosed method for on-site sanitizing or disinfection of an optical instrument eyepiece comprises placing a retention ring or gasket around the eyepiece and fixing the ring or gasket in place. A housing with an opening is placed over at least a portion of an eyepiece to be secured to the retention ring or gasket to light seal an inner cavity of the housing while providing space between interior of the housing from the eyepiece. The inner cavity of the housing has one or more UV-C LEDs and an inner surface comprising a UV reflective surface. A user initiates the irradiation of the cavity with UV-C light via a least one or more UV-C light sources positioned in the interior cavity.

As described in greater detail herein, the device is specifically adapted and designed to solve the problems with in-situ disinfection of optical device eyepieces. A common method of cleaning optical device eyepieces while they are on site in a laboratory, for example, includes the use of an alcohol-based wipe for metal surfaces and a lint free cotton swab for the optical surfaces. In the common cleaning method example, since the cleaning is done by hand, and is required to be completed often, cleaning may be done by different users in a potentially non-consistent manner. The device can minimize or eliminate the need for chemical cleaners that may include hazardous chemicals or chemicals that have a deleterious effect on the surface of optical device eyepiece providing a safer way to use the optical devices and providing a greater consistency in disinfection.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a view of two of the disclosed UV disinfection devices placed over two microscope eyepieces;

FIG. 2 is a view of a disclosed device exploded away from a microscope eyepiece showing the direction of attachment to a microscope eyepiece;

FIG. 3 is a cross sectional view of cross section of lines 3-3 of FIG. 2 of the disclosed device attached to an eyepiece;

FIG. 4 is a cross sectional view of a cross section of the UV disinfection device showing an alternate arrangement of an LED light source within the device;

FIG. 5 is a front perspective view of the UV disinfection device;

FIG. 6 is a rear perspective view of the UV disinfection device;

FIG. 7 is an exploded bottom perspective view of UV disinfection device with the electronics housing exploded from the cylindrical shroud and retention ring;

FIG. 8 is a bottom view of the UV disinfection device;

FIG. 9 is an exploded top perspective view of the components of electronics housing of the disclosed device;

FIG. 10 is an exploded bottom perspective view of the components of the electronics housing of the disclosed device;

FIG. 11 is a top perspective view of the retention ring of the disclosed device;

FIG. 12 is an exploded perspective view of the components of the retention ring; and

FIG. 13 is a view of the disclosed UV disinfection device exploded away from a microscope eyepiece showing direction of attachment to the retention ring.

### DETAILED DESCRIPTION

The detailed description set forth below in connection with the appended drawings is intended as a description of certain embodiments of a microscope eyepiece sanitizing mechanism and method of use and is not intended to represent the only forms that may be developed or utilized, nor are the described methods the only methods that could be employed. The description sets forth the various structure and/or functions in connection with the illustrated embodiments, but it is to be understood, however, that the same or equivalent structure and/or functions may be accomplished by different embodiments that are also intended to be encompassed within the scope of the present disclosure. It is further understood that the use of relational terms such as first and second, and the like are used solely to distinguish one entity from another without necessarily requiring or implying any actual such relationship or order between such entities.

In some embodiments, the numbers expressing dimensions, quantities, quantiles of ingredients, properties of materials, and so forth, used to describe and claim certain embodiments of the disclosure are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the disclosure are approximations, the numerical values set forth in the specific examples are reported as precisely as practicable. The numerical values presented in some embodiments of the disclose may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the disclosure and does not pose a limitation on the scope of the claimed inventive subject matter. No language in the specification should be construed as indicating any non-claimed element essential to the practice of the inventive subject matter.

Groupings of alternative elements or embodiments of the inventive subject matter disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed. Various objects, features, aspects and advantages of the inventive subject matter will become more apparent from the following detailed description of preferred embodiments, along with the accompanying drawing figures in which like numerals represent like components.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

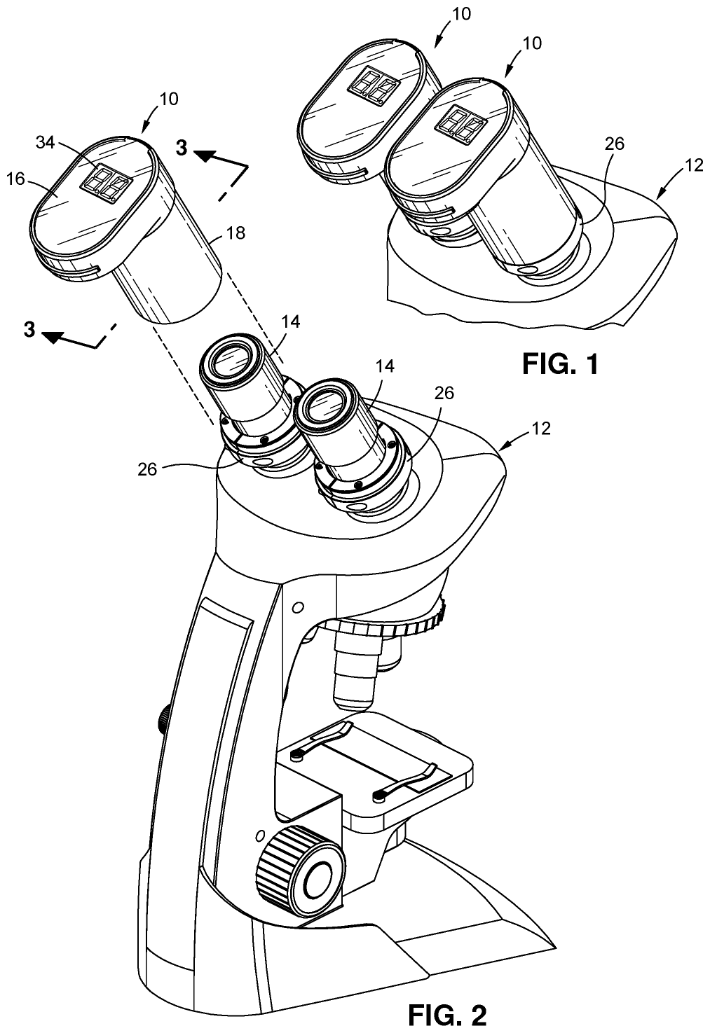

Referring particularly to FIGS. 1 and 2, there is shown the eyepiece sanitizing device 10 of the present disclosure in the environment of interfacing with a microscope 12. For example, in FIG. 1 the device 10 is shown mounted to both eyepieces 14 (shown in FIG. 2) of the microscope 12. In the embodiment shown in FIGS. 1 and 2 herein, the devices 10 are shown as =single units, however it is contemplated by this disclosure that a dual unit device may be constructed having the same components as device 10 but physically interconnected to form a single physical unit with two devices 10. Alternatively, it is contemplated by this disclosure that such a dual unit may have two units, but the units could be in electrical communication or in radio communication, such as Bluetooth®, and share electronics so that control electronics may be on board only one of the devices, but would control operation of both devices. For example, if the units worked in unison, only one LCD countdown display may be required. In both dual construction examples discussed above, the units may be interconnected by a fixed member to hold the units together, or interjoined by an adjustable member that allows for different spacing between device 10 units to fit microscopes that might have different spacing.

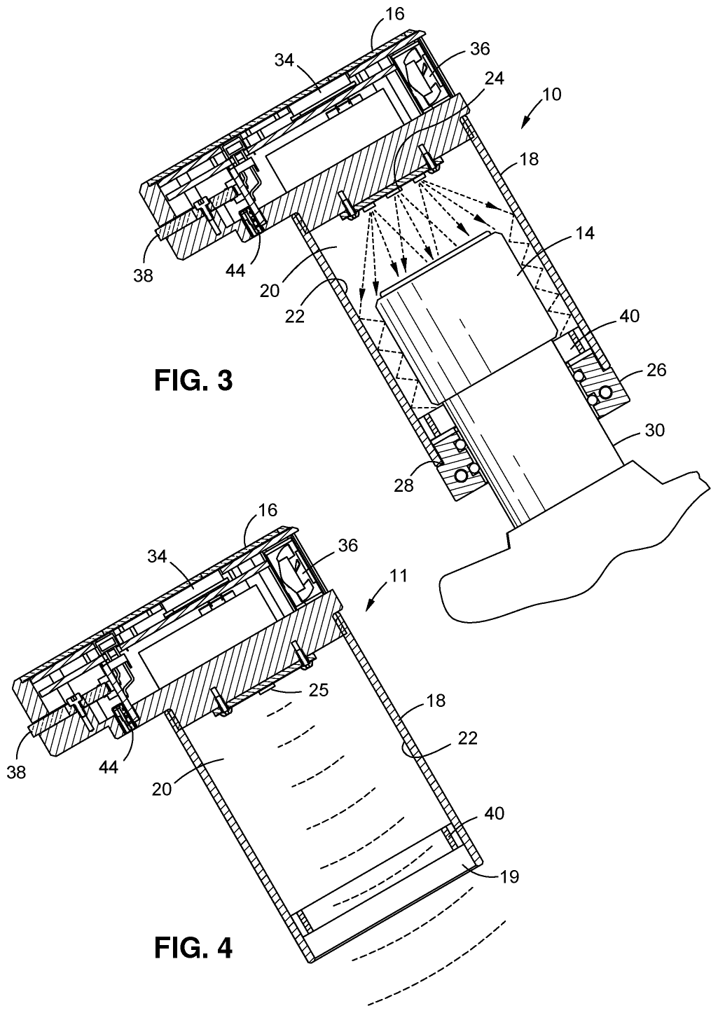

Referring to FIG. 2, there is shown the device 10 interfacing with an eyepiece 14 of a microscope 12, and the device 10 is shown exploded from the eyepiece 14, with dashed lines showing how the device 10 is received onto the eyepiece 14. FIG. 3 shows a cross section of the device 10 along lines 3-3 of FIG. 2 engaged with the eyepiece 14. The component parts of device 10 includes an electronics housing 16 and cylinder 18 that when connected together form the device 10 which defines an interior cavity 20 (see FIG. 3). Referring to FIG. 3, the cylinder 18 includes an interior surface 22 which includes a highly reflective surface. The reflective surface is suitable to reflect UV light and may be comprised of polished anodized aluminum; however any suitable reflective surface would be sufficient. Such reflective surfaces may also include polished nickel plated surface, a chrome plated surface, expanded PolyTetraFluor Ethylen (e-PTFE), Aluminum sputtered on glass, aluminum foil or various formulas of stainless steel. Stainless steel has the advantage of being resistive to microbial growth.

Again, referring to FIG. 3, the cross section of the device 10 affixed over an eyepiece 14 is shown. The cylinder 18 of the device 10 engages a retention gasket 26. Retention gasket 26 is secured to the outside of the eye tube 30 of the eyepiece 14 to provide a stop limiter so that the cylinder 18 does not move too far down the eyepiece 14 to leave enough space between the electronic housing 16 and the top of the eyepiece 14 to avoid blockage of UV light inside the interior cavity 20. Also, the cylinder 18 engages the lip 28 of the gasket 26 for frictional fit onto the gasket 26 and to hold the cylinder 18 inner surface 22 a proper distance from the eyepiece 14. In addition, the cylinder 18 and the gasket 26 provide a tight seal so as to prevent the leakage of UV light outside of the device 10 when in operation. In operation, an LED array 24 comprises three UV-C LEDs that emit UV-C radiation in or about the wavelength range of 220 nm to 300 nm. The LED array 24 provides UV radiation ranging from 220 nm to 300 nm, the wavelength range known to disrupt bacteria, viruses, protozoa, and/or the like. An example of a suitable UV-C LED is the UV AAP series (CUD8AF1D) deep UV LED sold by Seoul Viosys of the Republic of Korea. The LED array 24 emits UV-C. In FIG. 3, arrows indicate that emission of UV radiation when the LEDs of the LED array 24 are activated, and the device 10 is engaged with the gasket 26 so that there is sufficient space in the cavity 20 to permit the top of eyepiece 14 to be irradiated, and enough space between the inner surface 22 to the surface of the eyepiece 14 to permit UV light to reach all surface areas of the eyepiece 14 as demonstrated by the dashed lines with sharp turns in alternating directions between the sides of the eyepiece 14 and the inner surface 22 of the cylinder 18. Distribution of light in the cavity 20 ensures that that all surfaces of the eyepiece 14 within the device 10 are irradiated with a proper dosage of UV light to render viruses, bacteria and other pathogens harmless. Alternatively, the array 24 may comprise a single LED 25 as shown in FIG. 4. The dashed lines in FIG. 4 show the direction of emission of light from LED 25 if the LED were activated without an eyepiece 14 being received in the cylinder. The disclosure contemplates any suitable number of UV-C LEDs to be placed within the cavity 20 of the device 10.

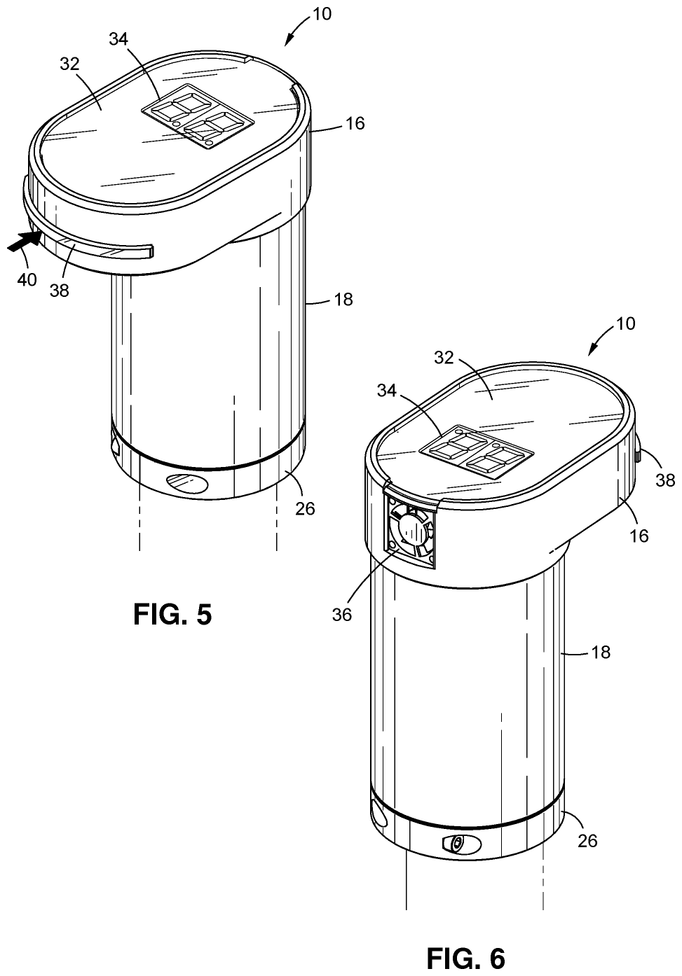

Referring particularly to FIGS. 5 and 6 there is shown a front (FIG. 5) and rear (FIG. 6) views of the device 10 connected to the retention gasket 26. The device includes a top electronics housing 16 and cylinder 18. On the upper face 32 of the electronics housing 16 a LCD digital readout 34 which provides a digital timer readout to the user for operation of the device. In operation a user may program the device 10 for the number of seconds or minutes for the LED array 24 to activate to provide UV light and the time remaining may be shown on readout 34 as it counts down the remaining time. Although the device 10 has an activation time of 60 seconds, it is contemplated by this disclosure that any suitable time may be programed into the device to provide an effective dosage of UV light. A light bar 38 is formed of a light transmissive material such as a translucent plastic so that an internal LED may transmit light through the light bar 38 as an indicator to provide visual cues to the user. For example, the light bar may light up in an intermittent or blinking fashion while a disinfection operation is running during the LCD countdown on LCD display 34, and then turn on to a continuous light up when the disinfection process is complete. Also, the light bar may change colors as a visual cue. This prevents the user from interrupting the process during use and also alerts the user when the process is complete. This disclosure also contemplates that the device 10 may include audible cues such as beeps or buzzes, for example, from appropriately positioned speakers or other audio generating devices. The light bar 38 also serves as a button to active the device 10. A user may depress the bar 38 inwardly in the direction of the arrow 40 to activate the device 10. Because the UV-C spectrum may be harmful to humans, when exposed to UV light to high intensity doses or exposure over a long period of time, the device 10 may include a flight sensor technology (not shown) that senses when the device is positioned at an angle or orientation that could cause accidental exposure of a user to UV light and therefore deactivates the device 10, disables the light bar 38 to act as an activation switch to interrupt or otherwise prevent the system from starting an activation or exposure cycle. The most sensitive human organs to UV-C light is the eyes and the skin to a lesser degree. Other types of sensors can be used to render to avoid user exposure to UV light including capacitance sensors, gyroscopic, and motion sensors.

A fan 36 is incorporated into the housing 16 which is activated for the sixty second period of operation of the LED array 24 in order to dissipate heat. It is additionally contemplated by the disclosure that the fan may be activated by a temperature sensor upon the internal temperature of the device 10 reaching a threshold temperature. The device 10 also includes an over temperature sensor (not shown) that shuts down device 10 if the temperature reaches a threshold level. The device 10 and the gasket 26 shown in FIGS. 5 and 6 show the tight fit of the cylinder 18 onto the lip 28 of the gasket 26 to seal the cavity 20 to mitigate UV light leakage. It is contemplated by the disclosure herein that the device 10 may have a connection sensor (not shown) such that a user may not be able to activate a UV radiation cycle if the device 10 is not securely attached to the gasket 26 to avoid accidental exposure of the user to UV light.

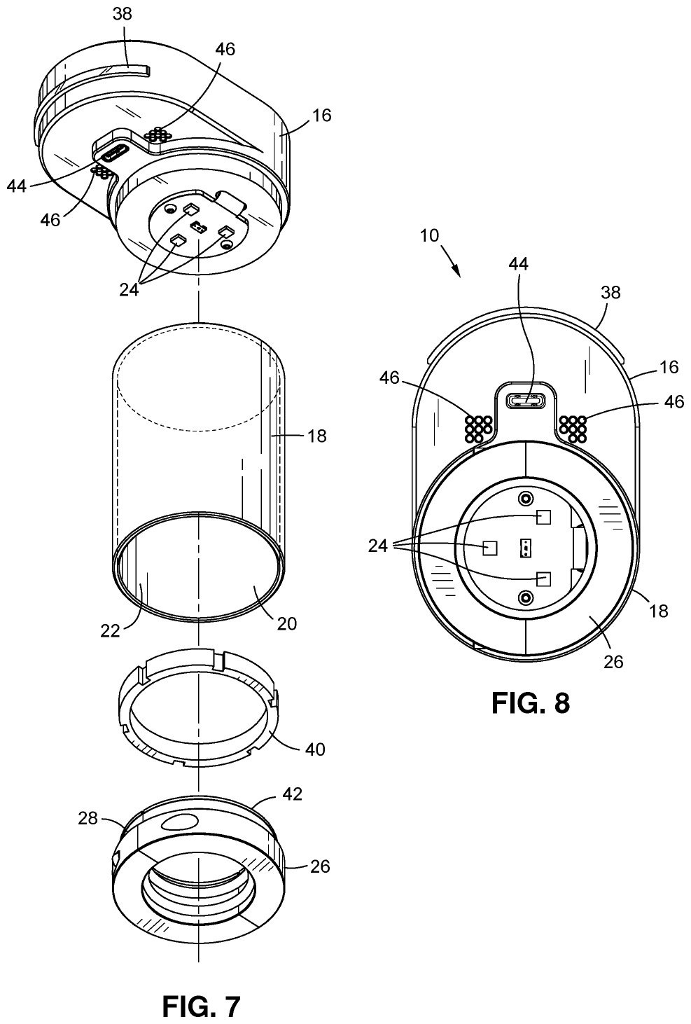

Referring particularly to FIG. 7 there is shown an exploded view of the device 10 and the retention gasket 26 demonstrating the assembly of those components. The electronic housing 16 includes the exposed LED array 24 which when coupled to the cylinder 18 forms a cavity 20 (not shown) and the LED array 24 is exposed to the cavity 20. A collar 40 is inserted within the cylinder 18 at a depth equal to the distance between the lip 28 to inner end of the 42 of the retention gasket 26. The collar 40 may be formed of aluminum or other rigid material such as metal or plastic.

Referring to FIG. 8 there is shown a plan bottom view of the device 10 connected to the retention gasket 26 as shown in FIGS. 5 and 6. The bottom view of the device 10 shows a mini-connector port 44 which may be a USB-C connector. The system is powered from a common 110-220V 50/60 Hz power source available world-wide and connected to the port 44. Additionally, the system can be remotely powered via a battery pack or an internal rechargeable battery. It is additionally contemplated by this disclosure that an internal battery may be recharged by an induction coil. Also shown in FIG. 8 are vent holes 46 that allow for the passage of air to act as a heat mitigation and dissipation.

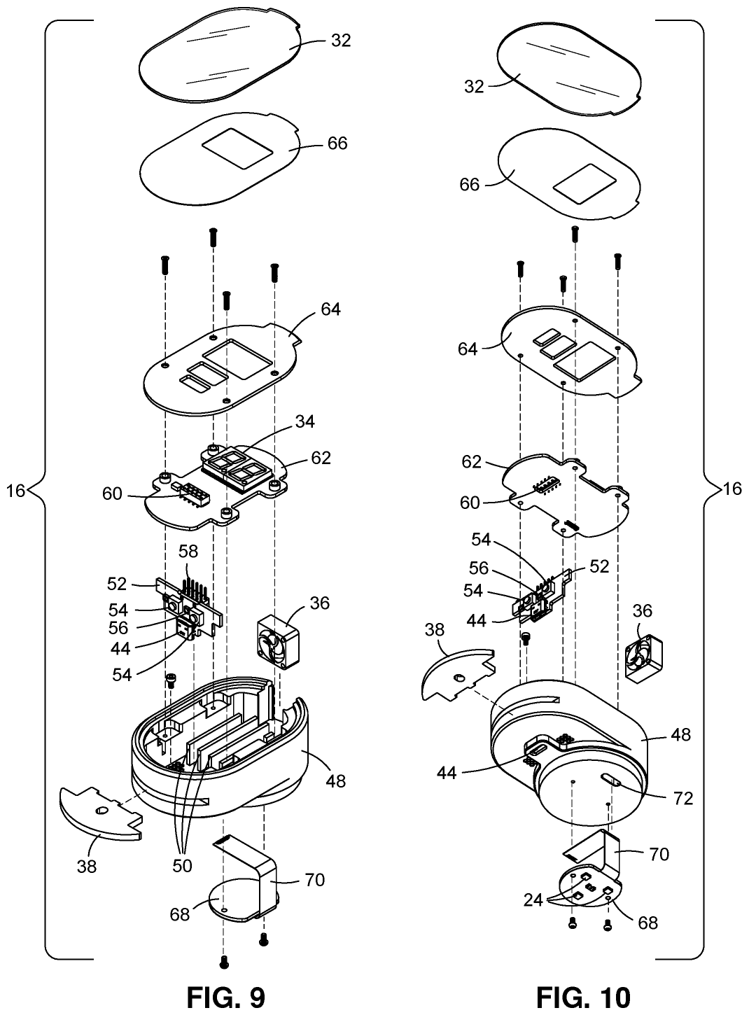

Referring particularly to FIGS. 9 and 10 there is shown exploded views of the electronics housing 16 of the device 10. FIG. 9 shows a top exploded perspective view and FIG. 10 shows an exploded bottom perspective view. The largest component of the housing 16 is the housing body 48 which if formed from solid metal such as aluminum or other suitable hard metal. The housing body 48 being formed of metal provides structural rigidity but also acts as a heat sink to aid in avoiding over heating of the LED array 24 during long exposures or activations. Cooling fins 50 are formed integrally to the housing body 48 to dissipate heat. As described above, a fan 36 is received into a slot in the housing body. The fan 36 is electrically connected to a control board 52 that actives the fan 36 via onboard electronics. The control board 52 is received within the housing body 48 in a vertical orientation. The bottom of the board 52 incorporates the port 44 which extends through the housing body to be accessible through the housing body 48. On the front face of the board 52, two pressure buttons 54 are provided to engage with the light bar 38, such that a user pressing upon the light bar 38 depresses the pressure buttons 54 to activate or deactivate a sanitizing cycle. A display LED 56 is positioned between the pressure buttons to transmit light through the translucent light bar 38 to prove a user with a visual cue. The LED 56 is a standard colored LED indicator light. Connector pins 58 engage with connector port 60 interconnect electronics on board the vertical board 52 with electronics on board the horizontal board 62 which includes LCD display 34. A top closure 64 provides support for the top surface 32 and includes openings to allow for viewing of the display 34 through a top flexible layer and top translucent surface 32.

The LED array 24 is positioned on an LED board 68. The LED board 68 is fastened to the housing body 48 via fasteners, and the LED board 68 is electrically connected to a lead 70 that is inserted into slot 72 formed into the housing body 48 and electrically connects with electronics on board the boards 52 and 62 wherein such onboard electronics drives the LED array 24. The LED array 24 is controlled by an electronic timer contained within the electronic housing 16 through a dedicated timer chip, or otherwise by a timing circuit or firmware of an on-board microprocessor. The LED array can comprise one or any number of LEDs that can fit within the structure. Also the LED board 68 is easily removable for and replaced for in-field repairs.

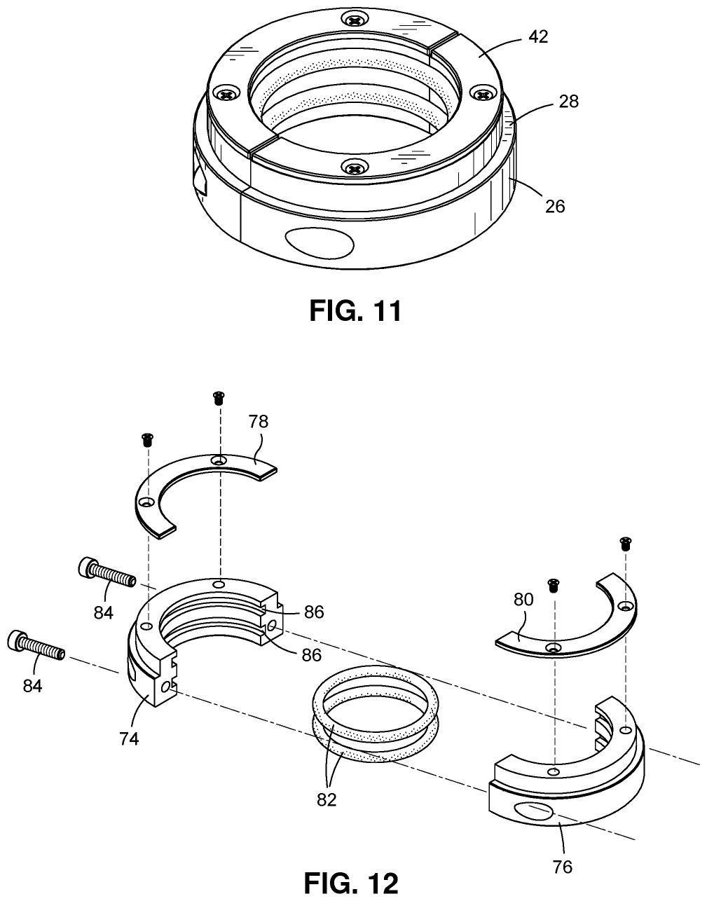

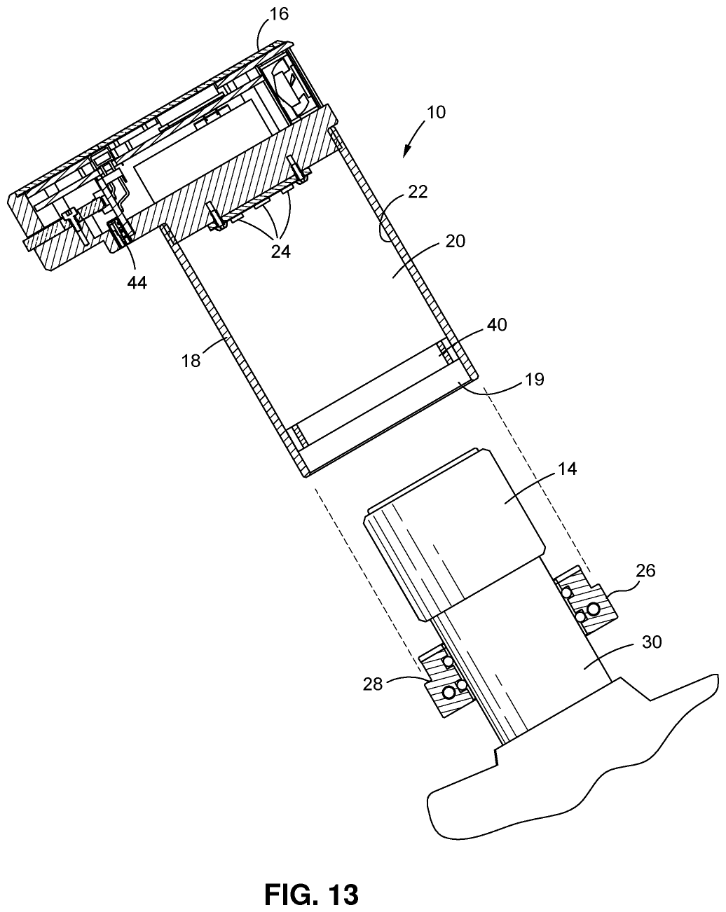

Referring to FIGS. 11-13 there is shown the retainer gasket 26 having an end surface 42 and lip 28, which is adapted to be attached to the tube 30 of an eyepiece 14. FIG. 12 shows an exploded view the gasket 26, and the various components for assembly. Referring to FIG. 12, the gasket 26 has gasket halves 74 and 76 which are connected via set screws 84 which pass through openings machined into the gasket halves 74 and 76. Ring plate halves 78 and 80 are formed from steel to form the top 42 and attached to the gasket halves 74 and 76 via fasteners. In operation, rubber rings 82 are slipped over the eye tube 30 and aligned with internal channels 86 formed on the inner curve of the gasket halves 74 and 76. The halves 74 and 76 are then closed and attached via set screws 84. The rings 82 are formed from rubber or other soft or resilient material and may protect the eye tube from damage and also provides additional space for larger eye tubes. Once in place as shown in FIG. 13, the device 10 engages the gasket 26 for support and to create seal.

The solution described herein can provide a safer portable design for in-situ sanitization of eyepiece surfaces, a common area for collection of viruses and bacteria. A longer operating lifetime (e.g., ultraviolet light emitting diodes can have a longer operating life than a typical mercury lamp), and more effective control of ultraviolet radiation parameters (e.g., wavelength, power, exposure time, radiation area, and/or the like). To this extent, the solution described herein can achieve an improved non-chemical sterilizing efficiency based on a specific absorption spectra of targeted bio structure(s). Also, the system is flexible enough to be adapted to all eyepieces used on microscopes and telescopes regardless of the manufacturer.

The organism(s) that can be disinfected by the UV-C light employed by the device and system can comprise any combination of various types of organisms, such as bacteria, viruses, protozoa, biofilms, mold, and/or the like. The discussion herein refers to the sterilization of all surfaces. As used herein, “sterilizing” and “sterilization” refer to harming one or more target organisms, and include purification, disinfection, and/or the like. Furthermore, as used herein a “sterilized surface” includes a surface that is devoid of any live organisms, a surface that is devoid of any live targeted organisms (but which may include non-targeted organisms), and a surface that includes some live targeted organism(s), but which is substantially free of such organism(s).

Telescopes, for example, typically have only one eyepiece and therefore a single device 10 that covers a single eyepiece would be effective. Microscopes, however, have either one or two eyepieces depending on the design. The single eyepiece device 10 will accommodate one eyepiece at a time allowing the operator to place the device into position and start a cleaning cycle. Once completed the will be moved to the second eyepiece and cycle would start again. With more than 90% of microscopes in place today having binocular heads using two eyepieces, it is contemplated by this disclosure to have a dual eyepiece version of the device 10. The disclosure contemplates synchronized cleaning between at least two devices 10. Devices 10 may be in electrical communication via connector or via radio signals such as Bluetooth and that the devices could be coordinated by a push of a button that sends a signal to one or more device 10s. Also, a Bluetooth connection may allow for control of the device 10 via a smartphone software application, and the processor and/or software of the smartphone software application might send instruction to one or more of the devices 10 to control the disinfection cycle and may control the device 10 independent of device 10 onboard electronics. Consider for example that a laboratory with a large number of microscopes wherein a technician places the device 10 on numerous microscopes and after placement initiates a cycle on a single device 10, and via radio communication such as Bluetooth the sanitation cycle is coordinated by the devices 10 intercommunicating. Likewise, consider a lab technician coordinating and setting the irradiation time of the devices 10 in the described multiple microscope example through a smartphone software application for a coordinated cycling for a group of microscopes. Additionally, in an application using a smartphone, the processor and memory of the smartphone may record the number of sterilizations completed by a particular device and other sterilization data as needed. In addition, the smartphone application may track or provide alerts when the disclosed device reaches a threshold number of uses so that the device can be serviced and/or components replaced.

The above description is given by way of example, and not limitation. Given the above disclosure, one skilled in the art could devise variations that are within the scope and spirit of inventive subject matter disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. An apparatus for sanitizing an optical instrument eyepiece comprising:
an elongate housing having a closed proximal end and an open distal end, the housing defining an interior cavity having an inner surface;
a UV reflective material formed on at least a portion of the inner surface;
at least one UV-C light source positioned on the elongate housing to direct light into the interior cavity; and
a retention gasket releasably secured to an outer surface of an eyepiece and adapted to be coupled to the distal end of the elongate housing.

2. The apparatus of claim 1 further comprising a controller in electrical communication with the at least one UV-C light source to provide a signal to activate and deactivate the at least one UV-C light source.

3. The apparatus of claim 1 further comprising a digital timer in electrical communication with the least one UV-C light source to provide a signal to activate and deactivate the at least one UV-C light source.

4. The apparatus of claim 3 wherein the digital timer is programmable.

5. The apparatus of claim 3 further comprising an LCD display in electrical communication with the digital timer.

6. The apparatus of claim 3 further comprising a manual switch in electrical communication with the digital timer.

7. The apparatus of claim 2 further comprising a sensor for detecting the orientation of the elongate housing, the sensor being in communication with the controller to provide a signal to the controller when the orientation of the elongate housing reaches a threshold angle.

8. The apparatus of claim 1 further comprising cooling fan attached to the elongate housing.

9. The apparatus of claim 1 wherein the UV-C light source comprises LEDs emitting light in the range of wavelengths between 220 nm to 300 nm.

10. A method for sanitizing an optical instrument eyepiece, comprising:
releasably attaching a retention gasket to the exterior of an optical instrument eyepiece;
receiving at least a portion of the eyepiece through an opening of a housing having an interior cavity comprising an inner surface, wherein the at least a portion of the inner surface comprises a UV reflective surface;
coupling the opening of the housing to the retention gasket to envelope the portion of the eyepiece within the interior cavity; and
irradiating the interior of the cavity with UV-C light via a least one UV-C light source positioned on the housing to direct light into the interior cavity.

11. A system comprising:
a UV-C based radiation source;
a shroud having an inner and outer surface, the inner surface comprised of a reflective material wherein the shroud is sized to cover at least a portion of the outside surfaces of an optical instrument eyepiece;
wherein the UV-C radiation source is positioned on the shroud to direct light to the inner surface of the shroud.

12. The system of claim 11, wherein the reflective material of the inner surface of the shroud is adapted reflect UV radiation to radiate surfaces of the eyepiece.

13. The system of claim 11 further comprising an adjustable collet adapted couple to an optical instrument eyepiece and to support the inner surface of shroud at a fixed distance from the surface of the optical instrument eyepiece to permit circumferential irradiation of the eyepiece surface.

14. The system of claim 11, wherein the UV-C irradiation source are LED having electronic controls that provide constant UV-C intensity over a predetermined time.

15. The system of claim 14 wherein the predetermined time is the time sufficient to provide a dosage of irradiation to destroy pathogens or render the pathogens harmless.

16. The system of claim 14, wherein the electronic controls comprise a timer in electrical communication with indicator LEDs that visually indicate that the system is ready to process, and when the system has completed an irradiating process over the predetermined time.

17. The system of claim 11, further comprising a safety sensor to sense the orientation of the shroud to provide a signal to a controller when the orientation is at a threshold angle.

18. The system of claim 13, wherein the collet is coupled to the shroud via a magnet.

19. The system of claim 11 further comprising a second system, wherein said first and second systems may be electronically coupled to engage with optical devices having two eyepieces.

20. The system of claim 19, wherein first and second systems may be controlled from one of the single systems.
